(19) United States
(12) Patent Application Publication
(10) Pub. No.: US 2026/0351341 A1
(43) Pub. Date: Nov. 5, 2026

(54) Systems and Methods for Optimizing Parameters for Targeted Cell Transduction Outcomes

(71) Applicants: Emory University, Atlanta, GA (US); Children's Healthcare of Atlanta, Inc., Atlanta, GA (US)

(72) Inventors: Reginald Tran, Atlanta, GA (US); Wilbur Lam, Atlanta, GA (US)

(21) Appl. No.: 19/484,740

(22) PCT Filed: May 15, 2024
(86) PCT No.: PCT/US2024/029455

§ 371 Date: Nov. 14, 2025

Related U.S. Application Data
(60) Provisional application No. 63/466,468, filed on May 15, 2023.

(51) Int. Cl. G16B 20/10; G16B 45/00

(52) U.S. Cl. CPC G16B 20/10; G16B 45/00

(57) ABSTRACT

The devices, systems, and methods may include can provide optimization and standardization of transduction parameters. The method may determine optimized transduction parameters. The method may include receiving (i) specifications for one or more transduction outcomes; and (ii) bulk transduction outcomes for one or more transductions of cells with a viral vector using at least one transduction parameter. The specifications may include a target number of vector integrations per cell. The method may include generating a model for each bulk transduction outcome to determine each bulk transduction outcome associated with a range of values of the at least one transduction parameter. The method may include generating distributions of vector integrations per cell for each value of the transduction parameter using the associated modeled bulk transduction outcomes. The method may also include determining an optimized transduction parameter for each transduction parameter from the distributions of vector integrations based on the specifications.

### CROSS-REFERENCE TO RELATED APPLICATIONS

This application claims the benefit of U.S. Provisional Application No. 63/466,468 filed May 15, 2023. The entirety of this application is hereby incorporated by reference in its entirety for all purposes.

### STATEMENT REGARDING FEDERALLY SPONSORED RESEARCH OR DEVELOPMENT

This invention was made with government support under HL129141 awarded by the National Institutes of Health. The government has certain rights in the invention.

### BACKGROUND

Conventional transduction methods, for example, for cell therapies, generally involve adding viral vector to target cells. Transduction outcomes for a given amount of viral vector can be extremely difficult to determine and generally can only be assessed several days after a transduction, for example, by measuring transduction efficiency (i.e., percentage of gene-modified cells) using flow cytometry or average copy number (VCN) (i.e., the number of integrated vector copies in the host cell genome) by quantitative polymerase chain reaction (qPCR) or droplet digital polymerase chain reaction (ddPCR). In an attempt to maximize gene transfer, excessive amounts of viral vector are often used. However, excessive viral vector usage can result in either waste if the majority of the viral vector is unable to reach and infect the target cells, or can carry an increased risk of insertional mutagenesis if cells undergo multiple vector insertions. Both scenarios can also occur simultaneously, posing safety concerns to patients and viral vector capacity shortages in cell therapy manufacturing.

One approach to describe how much viral vector is used in a transduction and/or to estimate how much vector might be sufficient to reach the minimum therapeutic threshold for a cell type is determining multiplicity of infection (MOI). However, in practice, MOI often far exceeds the average VCN, highlighting the waste of viral vector. Thus, MOI has been insufficient to quantifiably predict transduction outcomes.

### SUMMARY

Thus, there is a need for methods and systems that can accurately and quantifiably predict transduction parameters, such as vector dosage, without needing excessive viral vector usage.

The methods and systems of the disclosure can improve transduction outcomes without needing excessive viral vector usage and can therefore improve patient safety, provide a means to reduce patient variability, enable tuning of therapeutic efficacy, and reduce the amount of wasted viral vector.

In some embodiments, the methods include a method for determining optimized transduction parameters. In some examples, the method may include receiving (i) specifications for one or more transduction outcomes; and (ii) bulk transduction outcomes for one or more transductions of cells with a viral vector using at least one transduction parameter. The specifications of one or more transduction outcomes may include a target number of vector integrations per cell. The one or more bulk transduction outcomes may include average vector copy number and transduction efficiency. The method may include generating a model for each bulk transduction outcome to determine each bulk transduction outcome associated with a range of values of the at least one transduction parameter. The method may also include generating distributions of vector integrations per cell for each value of a number of values of the at least one transduction parameter using the associated modeled bulk transduction outcomes. The method may further include determining an optimized transduction parameter for each transduction parameter from the distributions of vector integrations based on the specifications.

In some examples, a value of the at least one transduction parameter for each transduction may be different. In some examples, each optimized transduction parameter may correspond to a value of each modeled transduction parameter from the distributions that meets the specifications. In some examples, the at least one transduction parameter including a dose of the viral vector.

In some examples, the methods may include a method for generating a standardized visualization of transduction outcomes. The method may include providing a standardized relationship between transduction efficiency and average VCN with respect to integration distributions. The method may also include receiving one or more sets of bulk transduction outcomes determined for one or more viral vectors and/or for one or more cell manufacturing processes. The method may also include mapping each set of bulk transduction outcomes to the standardized relationship. The method may further include generating a visualization of the mapping.

In some examples, the standardized relationship may be depicted as a curve bound plot. Each set of bulk transduction outcomes may be for a different viral vector.

In some examples, the one or more sets of bulk transduction outcomes may be for previously used cell manufacturing processes and the one or more sets of bulk transduction outcomes are for candidate cell manufacturing processes. In some examples, the mapping may include juxtaposing the one more sets of bulk transduction outcomes for the candidate cell manufacturing processes to the one or more sets of bulk transduction outcomes for previously used cell manufacturing processes on the standardized relationship.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1 shows a flow chart illustrating an example of a method of determining transduction parameters according to embodiments;

FIG. 2 shows a flow chart illustrating an example of a method of determining a vector integration distribution according to embodiments.

FIGS. 3A-E show illustrative examples of optimizing a transduction dose using a batch of CD19-CAR LV with the same donor's cells. FIG. 3A shows an example of a model of transduction efficiency generated according to embodiments. FIG. 3B shows an illustrative example of a model of average VCN generated according to embodiments. FIG. 3C shows an illustrative example of generated model of distributions and specifications to determine optimized transduction parameter(s). FIG. 3D shows an illustrative example of a comparison of the transduction efficiency using the optimized transduction parameter in a scaled-up device. FIG. 3E shows an illustrative example of a comparison of the average VCN using the optimized transduction parameter in a scaled-up device.

FIG. 4 shows an illustrative example of a pooled distribution generated according to embodiments.

FIG. 5 shows an example of method of generating a visualization of transduction outcomes mapped to a standardized space based on integrations according to embodiments.

FIG. 6 shows an illustrated example of a visualization of the mapping of transduction outcomes generated according to embodiments.

FIGS. 7A and B show illustrative examples of an output of the visualization of the mapping of the bulk transduction outcomes to the standardized space generated according to embodiments. FIG. 7A shows an output of the visualization of mapping of the bulk transduction outcomes on a standardized space. FIG. 7B shows a visualization of the associated integrations for specific points identified within the space shown in FIG. 7A.

FIG. 8 shows another illustrative example of an output of the visualization of the mapping of the bulk transduction outcomes for a plurality of different cell products to the standardized space according to embodiments.

FIGS. 9A-C show illustrative examples of an output of the visualization of the mapping of transduction outcomes for a plurality of different cell vectors according to embodiments. FIG. 9A show an output of the visualization of mapping the bulk transduction outcomes of different viral vectors to the standardized space according to embodiments. FIG. 9B shows a visualization of the transduction efficiencies for respective viral doses associated with each vector mapped to the output shown in FIG. 9A. FIG. 9C shows a visualization of the average vector copy number for respective viral doses associated with each vector mapped to the output shown in FIG. 9A.

FIG. 10 shows an illustrative example of an output of the visualization of the mapping of transduction outcomes for a plurality of different cell manufacturing processes according to embodiments.

FIG. 11 shows a block diagram illustrating an example of a system according to embodiments.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The systems and methods of the disclosure can provide optimization and standardization of transduction parameters, enabling more conditions to be screened with lower reagent consumption. The systems and methods can accurately and quantifiably characterize transduction outcomes and therefore can improve safety, can provide a means to reduce patient variability, enable tuning of therapeutic efficacy and reduce the amount of wasted viral vector. Ins some examples, the systems and methods can generate a distributions of vector integrations per cell, for example, using bulk transduction outcomes, such as average VCN and transduction efficiency. The distributions of vector integrations per cell can provide an objective characterization of transduction outcomes, and when assessed for multiple transductions and linked to the associated transduction inputs, allows for individualized transduction dynamics of a specific cell and transduction parameters, such as viral dose, to be characterized. In some examples, this can enable a determination of optimized transduction parameter(s), such as vector that can maximize transduction efficiency while minimizing the average VCN. In some examples, the optimized transduction parameter(s) can be used in manufacturing cell-based products, studying cell-based products, as well as personalized manufacturing of cell-based products.

By determining a model of distributions of vector integrations per cell, the disclosed systems and methods can determine transduction parameters that better comply with health agency's requirements or guidance. By way of example, the FDA and WHO currently recommend that vector integrations per cell should remain under five copies per cell to minimize risk of insertional mutagenesis. Current conventional transduction parameters generally only measure average VCN so it may not capture the entire cell population. For example, although average VCN may be less than five, there may be a large proportion of transduced cells with more than five vector integrations. Thus, the systems and methods can determine transduction parameter(s) that maximize therapeutic efficiency while minimizing risk of insertional mutagenesis.

The systems and methods can also provide an objective assessment of transduction outcomes for safety assessments according to health agency's recommendations or requirements. For example, the systems and methods can be used to generate a graphical representation that can provide a rapid visualization of the objective assessment by mapping the transduction outcomes to a standardized space representing all possible distributions of vector integrations per cell such that the determination of a transduction outcome's compliance with health agency's requirements or guidance can be quickly determined by its positioning within colored regions of the standardized space. This assessment can determine whether optimization of one or more transduction parameters should be performed.

In some examples, the systems and methods of the disclosure can determine a distribution of vector integrations per cell, a priori, for example, using generated models based on transduction outcomes determined from small-scale transductions and/or a model. By enabling a determination a priori, the systems and methods of the disclosure can address the deficiencies of the current a posteriori approach to evaluating transduction outcomes. This can enable transduction parameters to be tailored to manufacturer specifications, clinical study specifications, and patient specifications. For example, the systems and methods of the disclosure can determine a personalized transduction parameter(s) (i.e., viral vector dose) tailored to the patient's specific vector integration dynamics thereby enabling manufacture of the patient's cells to a certain specification.

In some examples, the systems and methods of the disclosure can enable a linearly scalable transduction platform that allows for transduction of a small number of cells to match transduction of a larger number of cells with high fidelity. This way, the systems and methods of the disclosure can determine transduction parameters that can achieve a target transduction outcome for pre-clinical or clinical-scale transductions from small-scale transductions.

The disclosed systems and methods can also improve treatment outcomes using less resources. For example, the optimized transduction parameters determined by the disclosed systems and methods can obviate the need for high statutory doses of viral vector and lengthy transduction times. This can, for example, improve cell engraftment and stem cell characterizations when transplanted to patients.

In some examples, the disclosed systems and methods can determine optimal transduction parameters that minimize donor variability. For example, the disclosed systems and method can determine optimized transduction parameters (e.g., vector dose) using a pooling of distributions of different cell donors.

In these ways, the disclosed systems and methods can address the historically challenging aspects of transduction. The disclosed systems and methods enable multiple conditions of transduction parameter(s) (e.g., viral vector dose, transduction time, enhancer concentration, etc.) to be simultaneously analyzed with reduced resources (e.g., reduced vector requirements and time needed to set up the transductions). By enabling a predictive and objective assessment of transduction outcomes, it can address the deficiencies of using MOI to predict transduction.

As used herein, the term “vector” refers to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Vectors can include, for example, plasmids (e.g., DNA plasmids or RNA plasmids), transposons, cosmids, bacterial artificial chromosomes, and viral vectors. Viral vectors can include but are not limited to retroviral vectors, lentiviral vectors, other integrating vectors, among others, or a combination thereof.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term “viral vector” or “VV” may refer either to a virus or viral particle capable of transferring a nucleic acid into a cell or to the transferred nucleic acid itself. Viral vectors and transfer plasmids contain structural and/or functional genetic elements that are primarily derived from a virus. The term “lentiviral vector” refers to a retroviral vector or plasmid containing structural and functional genetic elements, or portions thereof, including LTRs that are primarily derived from a lentivirus.

As used herein, “transduction” refers to the introduction of foreign nucleic acid molecule into a cell's genome using a viral vector.

As used herein, “vector copy number” or “VCN” refers to the number of copies of a vector (also referred to as vector integrations), or portion thereof, in a cell's genome. The average VCN may be determined from a population of transduced cells or from individual cell colonies.

“% Transduced cells” or “% TD” refers to the percentage of cells transduced with at least one copy of a vector. For example if 1×106 cells are exposed to a virus and 0.5×106 cells are determined to have a least one copy of a virus in their genome, then the transduction efficiency is 50%.

“Viral vector dose” (also referred to as “viral dose”) refers to a volume of viral vector used for a transduction. In some examples, the dose may be referred in units of μL VV/Cell.

“Integration distribution of transduced cells” refers to the collective percentages of cells consisting of integer vector integrations (ex. 0, 1, 2, 3, etc.) within the transduced cell population. The sum of all percentages of cells should equal 100%.

“Cell products” may be used in cell-based therapies in which the transduced or manufactured cells using viral vectors can be injected, grafted and/or or implanted into a patient.

Various embodiments are described herein, including systems, methods, devices, modules, models, algorithms, networks, structures, processes, computer-program products, and the like.

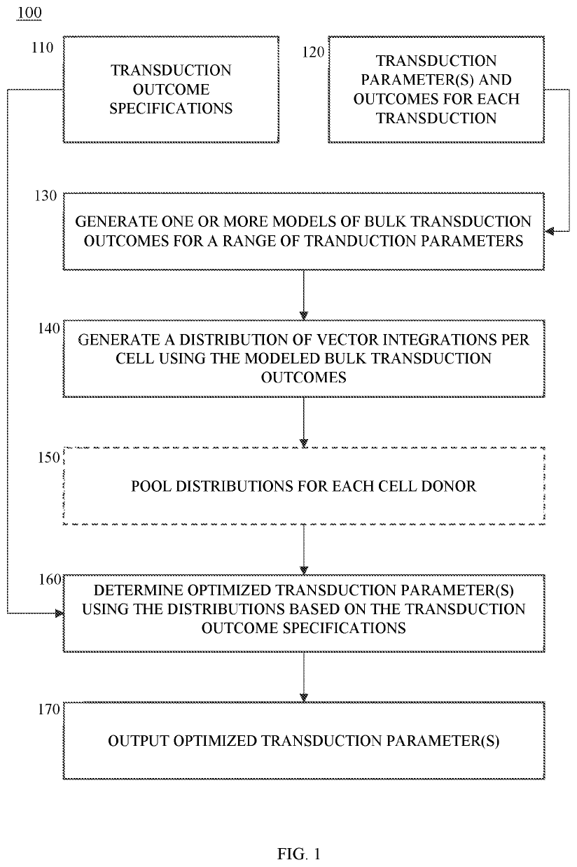

FIG. 1 shows an example of a flow chart 100 illustrating a method for determining one or more transduction parameters according to embodiments. The one or more transduction parameters may refer to transduction parameter(s) that is optimized for the target transduction outcomes, for example, as provided in the transduction outcome specification(s). In some examples, the one or more parameters may include but are not limited to viral vector dose, transduction time, reagents, concentration enhancer, number of cells, stimulation parameter (e.g., prior to transduction), device parameters (e.g., fluid height, surface area, etc.), cell concentration, among others, or a combination thereof. While the examples may be specific to determining viral vector dose, it will be understood that additional and/or alternative transduction parameters may be determined using the distribution of vector integrations per cell. For example, the one or more transduction parameters may additionally or alternatively include one or more of the other transduction parameters, among others, or a combination thereof.

Operations in the flow chart 100 may begin at block 110 and/or at block 120. At block 110, the specification(s) of one or more transduction outcomes may be provided. For example, the user may set the specification(s) of one or more of the transduction outcomes, the specification(s) of one or more of the transduction outcomes may be pre-set, among others, or a combination thereof. The specification(s) may include the target transduction outcome(s) for the transduction(s) to be performed. In some example, the one or more transduction outcomes may include but are not limited to bulk transduction outcomes, other transduction outcomes, or a combination thereof. Bulk transduction outcomes may include but are not limited to transduction efficiency and average VCN. In some examples, the other transduction outcome(s) may include but is not limited to percentage of cells with a number of integrations, viral dose, donor variability (e.g., a coefficient of variation (e.g., less than x %, such as 20%), among others, or a combination thereof. For example, the specification(s) may include a target value, such as a minimum or maximum value or range of values of one or more the transduction outcome(s). In some examples, the specification(s) may correspond to health agency's recommendations or requirements for transduction outcome(s) of cell-based therapies. For example, the percentage of integrations may correspond to FDA or WHO's recommendations and/or requirements. By way of example, the specification(s) s may include a transduction efficiency greater or equal to a value (e.g., 10%); average VCN within a range of values (such as greater than or equal to 1 but less than 5), and percentage of cells with 5+ integrations may be less than or equal to a value (e.g., 4%).

Although block 110 is shown as starting the operations in the flow chart 100, it will be understood that the specification(s) may be provided later in the operation. For example, the specification(s) may be provided with respect to the operations performed at block 150.

At block 120, transduction parameters and bulk transduction outcomes for one or more transductions may be received. In some examples, a number of small-scale transductions may be performed using different values of the transduction parameter to be optimized, such as viral vector dose, and/or using different cell donors, and the outcomes may be measured for each transduction. For example, at least three transductions may be performed using different viral doses. In this example, the viral vector dose and the associated transduction outcomes (e.g., transduction efficiency and average VCN) may be received. In further examples, transductions may be performed using different cell donors. In this example, the viral vector dose and associated transduction outcomes for each cell donor may be received.

In some examples, the transduction(s) may be performed using standard cell culture platforms, such as well plates, tissue culture flasks, cell culture bags; large-scale transduction devices, such as bioreactors; among others, or any combination thereof. In some examples, the transduction(s) may be performed using small-scale transduction devices, such as those disclosed in concurrently filed International Patent Application entitled “Systems and Devices for High Throughput Cell Vector Co-Localizations” with the docket number 22103 PCT, which is incorporated herein by reference; thereby enabling a determination of candidate transduction parameter(s) for large-scale production or patient-specific production of cell therapies without wasting viral product.

In some examples, one or more of the transduction outcomes may be determined using a model that associates viral vector doses to transduction outcomes. The model may be a mathematical model (e.g., regression model) or trained machine-learning model. For example, a model may be trained using data for a plurality of input transduction parameters such as vector dose, transduction time, enhancer concentration, etc., to model the transduction outcomes associated with vector doses. In this example, the associated transduction outcomes for block 120 can be determined for the associated viral dose using the model without physically performing the transduction.

Next, at block 130, one or more models of bulk transduction outcomes for a range of values of one or more transduction parameters may be generated for each bulk transduction outcome. If data for multiple cell donors are received at block 120, one or more models of bulk transduction outcomes for a range of one or more transduction parameters may be generated for each bulk transduction outcome for each cell donor.

Using these models, the transduction outcomes for a range of at least one transduction parameter (e.g., such as viral vector dose) may be predicted. In some examples, the one or more models may include one or more multivariate models (e.g., a model with multiple independent variables) or more than one models (e.g., models with a single independent variable).

In some examples, the one or more models may be generated using statistical learning or machine learning techniques. For example, the one or more models may be a regression model. The regression model may include but is not limited to linear regression, non-linear regression, among others, or a combination thereof.

In some examples, a model may be generated for each bulk transduction outcome received at block 120. For example, if viral vector dose as the transduction parameter and transduction efficiency and average VCN as the bulk transduction outcomes were received at block 120, one or more models may be generated associating the transduction efficiency and the average VCN with a range of viral vector doses. For example, a non-linear regression model may be generated for each bulk transduction outcome so there may be 1) a non-linear regression model modeling transduction efficiencies with respect to vector dose and 2) non-linear regression model modeling average VCN with respect to vector dose.

In some examples, the model may be generated using nonlinear regression, for example, by fitting the data to a model representative of the transduction dynamics. In this case, the data can be fitted to a hyperbolic curve, so that the relationship between an input vector dose and the bulk transduction outcomes provided at block 120 can be expressed as a hyperbolic function with the form Y=(Bmax*x)/(Kd+x), where Y is either % transduction or VCN, x is the input vector dose, and Bmax and Kd are the constants solved for by the respective nonlinear regression.

By way of another example, a multiple regression model may be generated for both vector dose and VCN. For example, a multiple regression model may be generated using partial least squares regression.

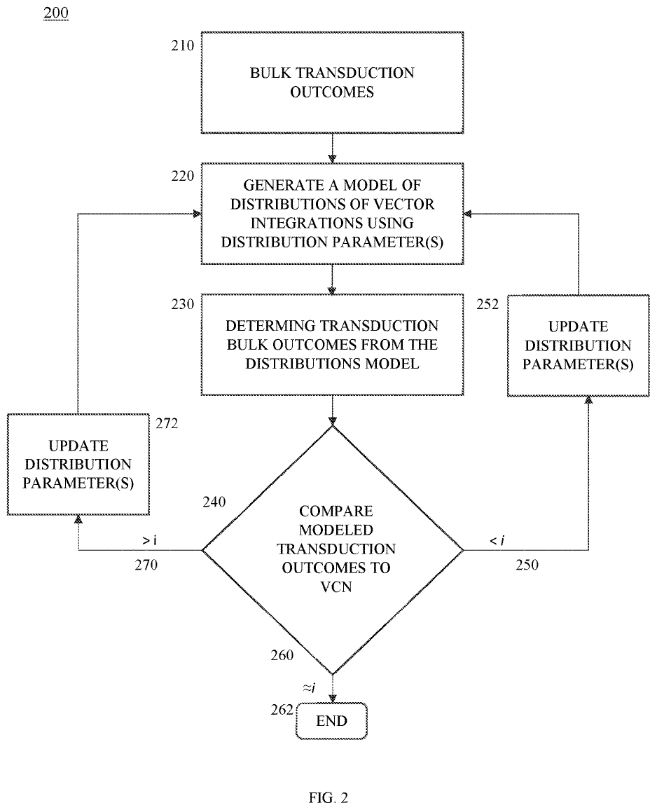

Next, at block 140, a model of distributions of vector integrations per cell for each value of a number of values of the at least one transduction parameter using the modeled bulk transduction outcomes may be generated. In some examples, a model of distributions may be generated using the modeled bulk transduction outcomes from block 130. In some examples, the model can provide a predicted relationship between a candidate transduction parameter (e.g., viral vector dose) and the vector integration distribution. In some examples, a model of distributions may be generated using Poisson distribution, other probability distributions, other discrete parametric probability distribution, among others, or a combination thereof. FIG. 2 shows an example of generating distributions of vector integrations according to some embodiments.

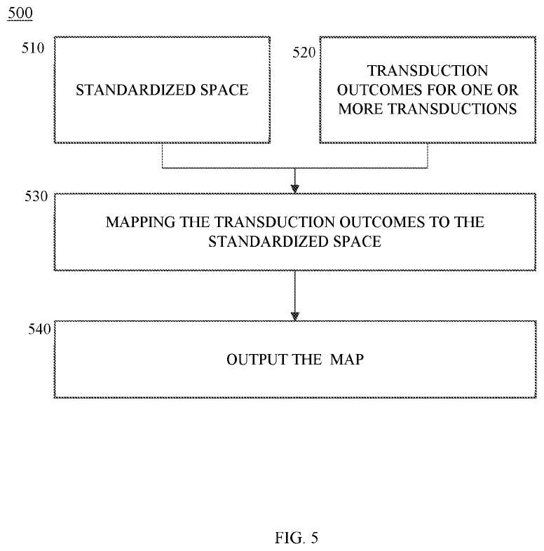

In some examples, if the method 100 is used to optimize transduction parameters for multiple cell donors, the operations at blocks 120-140 may be repeated for each cell donor to be evaluated. In this example, the method 100 may further include pooling the distributions for each donor to generate a pooled distribution at block 150. FIG. 5 shows an example of a pool distributions for cell donors. This can enable an objective evaluation of multiple donors to select transduction parameters (e.g., viral dose) based on specification(s) s of target transduction outcome(s) that address efficacy, safety, and minimizing donor variability. In this example, the optimal dose would correspond to the dose that provides the least variability between donors and satisfies the specifications.

Next, at block 160, the optimized transduction parameter(s) may be determined using the distributions from block 140 or pooled distributions from block 150 based on the transduction outcome specification(s) s from block 110. In some examples, the optimized transduction parameter(s) may correspond to a value of each modeled transduction parameter that meets the respective target(s) provided in the specification(s) s from block 110.

In some examples, operations at block 160 may be repeated for different transduction outcome specification(s) s provided at block 110. By way of example, optimized transduction parameter(s) may be determined from the distribution models generated at block 140 or pooled distribution models generated at block 150 for different outcome specification(s) s provided at block 110 without repeating the operations at blocks 120-150.

Next, at block 170, the optimized transduction parameter(s) may be outputted. For example, the optimized transduction parameter(s) may be transmitted to an automated cell culture system. In some examples, at block 170, the output may be causing the automated cell culture system to transduce cells according to the optimized transduction parameter(s). In some examples, the automated cell culture system may be a larger-scale device for transduction than the devices used for the initial transductions at block 120.

In some examples, one or more graphical representations of the distribution models and optimized parameter(s) may be generated and outputted at block 170. In some examples, the optimized transduction parameter(s) and/or graphical representation(s) may be stored and/or displayed.

In some examples, the optimized parameter(s) may be outputted with respect to the distributions in a graphical representation. For example, the graphical representation can be a mapping of outcomes parameter space for any transduction. In some examples, the graphical representation may be used for assessments of safety and/or performance.

FIG. 2 shows an example of a flow chart 200 illustrating a method for generating a model of distributions performed at block 140 according to embodiments.

Operations in the flow chart 200 may begin at 210, for example, when the bulk transduction outcomes are modeled at block 130. In some examples, different bulk transduction outcomes from performed transductions may be used. Next, at block 220, a model of distributions of vector integrations may be generated using distribution parameters and the bulk transduction outcomes, for example, from block 120. For example, if the model of distributions is an integration distribution (e.g., P(x,λ)) generated using a Poisson distribution, the distribution parameters may relate to lambda (λ) and x. In this example, lambda may relate to average rate of integrations per cell and x=number of integrations per cell. At block 220, the model of distributions may be generated using a preset value for lambda (e.g., for example 0.001). In some examples, the Poisson distribution can be represented as:

$$P ( x ) = \frac{{( 3.653 )}^{x}}{x !} {e}^{- ( 3.653 )} .$$

Next, at block 230, the transduction outcomes may be determined from the model of distributions generated at block 220. For example, the average VCN can be determined by dividing the summation of integrations by the number of cells. In some examples, for a known distribution P(x), the average VCN can be calculated using this expression

$${\sum }_{i = 1}^{∞} x \cdot P ( x ) .$$

Next, at block 240, the transduction outcome(s) from block 230 can be compared to the transduction outcome(s) from block 210. For example, the difference between the average VCN from block 210 and the average VCN from block 230 (e.g., VNC_{block 230}−VCN_{block210}) can be determined. The next operation can be based on that difference. In some examples, the difference may be compared to a threshold (i) to determine the next operation. In some examples the threshold (i) may correspond to 0. In other examples, i may have a different value or range of values.

If the difference is greater than the threshold (i), pathway 270, the distribution parameter(s) may be updated by decreasing the value of the distribution parameter(s) by a set amount at block 272. If the difference is less than the threshold (i), pathway 250, the distribution parameters may be updated by increasing the value of the distribution parameter(s) by a set amount at block 252. In some examples, if the model of distributions is generated by a Poisson distribution, the value of lambda may be updated. By way of example, if lambda was set at 0.001 at block 210, the value of lambda may be increased at block 250 and decreased at block 270 by 0.001.

If the difference equals the threshold, at pathway 260, then the operations may end at block 262 and the model of distributions generated at block 240, which can correspond to block 140, to optimize transduction parameters, for example, at block 160. In some examples, for example, when determining optimal parameters based on multiple cell donors, the distributions generated at block 240 may be pooled at block 150.

While some examples of the disclosure may be specific to determining viral vector dose using CD19-CAR LV, it will be understood that these example are nonlimiting and that the methods and systems may be performed for other viral vectors and cells.

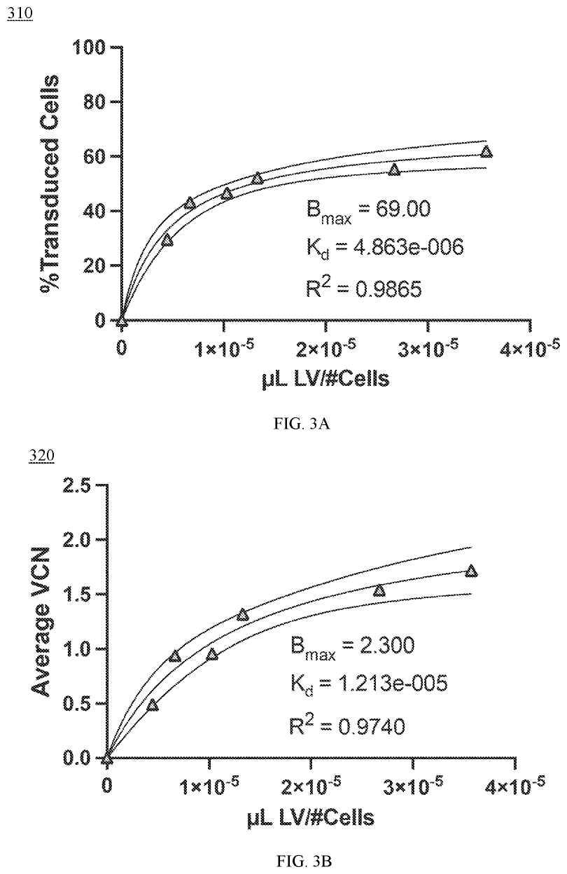

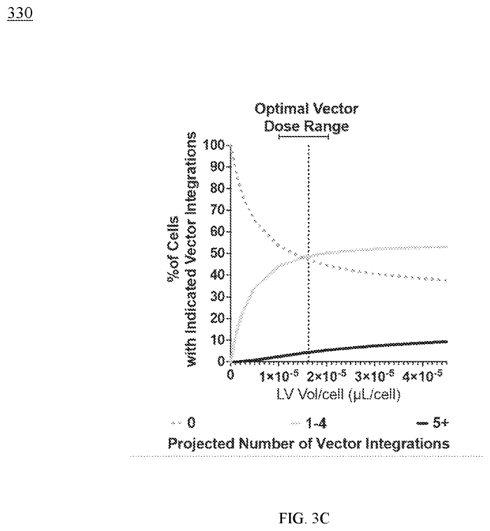

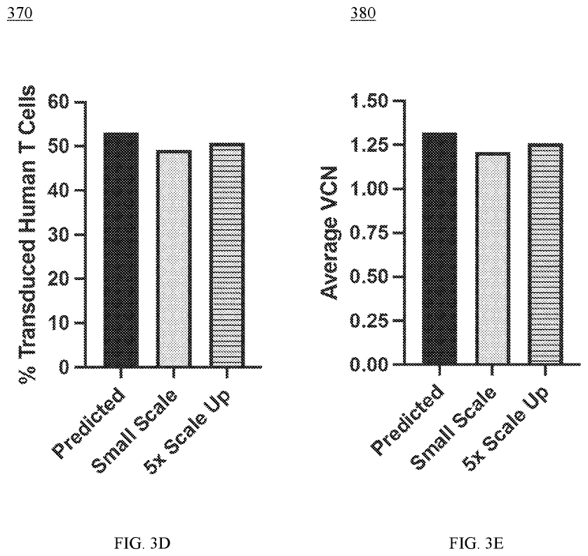

FIGS. 3A-E show illustrative examples of optimizing a transduction dose using bulk transduction outcomes measured for a batch of CD19-CAR LV with the same donor's cells, performed using a smaller scale device. In this example, the transduction was performed using a device similar to the devices described in the above incorporated International Patent Application entitled “Systems and Devices for High Throughput Cell Vector Co-Localizations” with the Attorney Docket Number 22103 PCT.

FIGS. 3A and 3B show illustrative examples 310 and 320, respectively, of transduction efficiency and average VCN in relation to input vector dose (μL LV/#cells) that were generated using the method described at block 130. For example, the models were generated by fitting the experimental transduction outcomes to a non-linear hyperbolic regression model. In these examples, the High R² values indicate that the data fit both models well and can provide accurate predictions of transduction outcomes for any given input vector dose for subsequent transductions when using this particular batch of CD19-CAR LV with the same donor's cells.

FIG. 3C shows an illustrative example 330 of a visualization of the distributions of vector integrations per cell generated using the models of FIGS. 3A and 3B by performing the operations at block 150 according to method 200 to determine the optimal viral dose, at block 160, that meets the specifications for a target outcome (from block 110). In this example, the specification for the transduction outcome may be the number of integrations that meet FDA's recommendations, such as having less than five integrations. In this example, the optimized parameter, the optimal vector dose range, corresponds to the viral dose resulting in transductions, such as the number of integrations, that meet the FDA recommendations.

FIGS. 3D and 3E show illustrative examples 370 and 380 of comparisons of the transduction outcomes from the initial transductions of a batch of CD19-CAR LV with the same donor's cell (e.g., block 120), the predicted transduction outcomes using the optimized viral dose shown in FIG. 3C, and the transduction outcomes from performing a larger scale transduction using the optimized viral dose. In this example, as shown, usage of the output optimized vector dose meeting the target specifications determined in FIG. 3C resulted in accurate targeting of % transduction (FIG. 3D) and average VCN (FIG. 3E) for both small scale transductions using the scalable high throughput transduction device and a larger 5× scale up channel-based device.

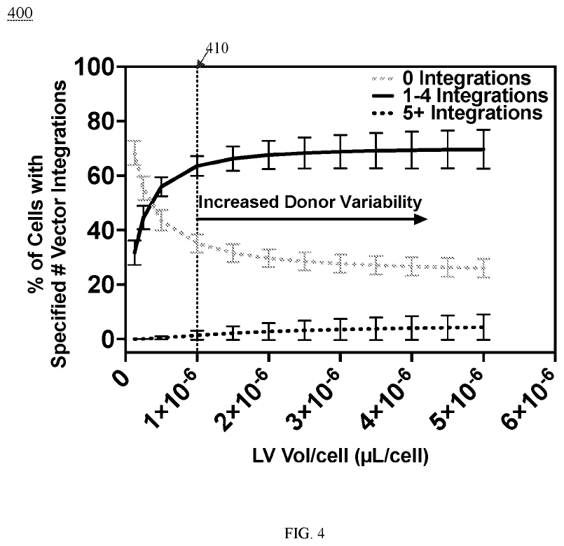

FIG. 4 shows an illustrative example 400 of pooled distribution, for example, generated at block 150. In this example, the distribution data for donors can be pooled so that the optimized transduction parameter can be selected. In this example, the optimal dose 410 can correspond to the dosage that provides the least variability between donors and satisfies the target specifications (e.g., at block 110).

FIG. 5 shows an example of a flow chart 500 illustrating a method for generating an objective assessment of outcomes with respect to integration distributions. In some examples, the objective assessment may be related to a standardized space. In some examples, the assessment may indicate that optimization of one or more transduction parameters should be performed, for example, according to embodiments (e.g., flow charts 100 and 200). In some examples, the assessment may be a map of the transduction outcomes to a standardized space representing the distribution of vector integrations per cell that comply with health agency's requirements or guidance.

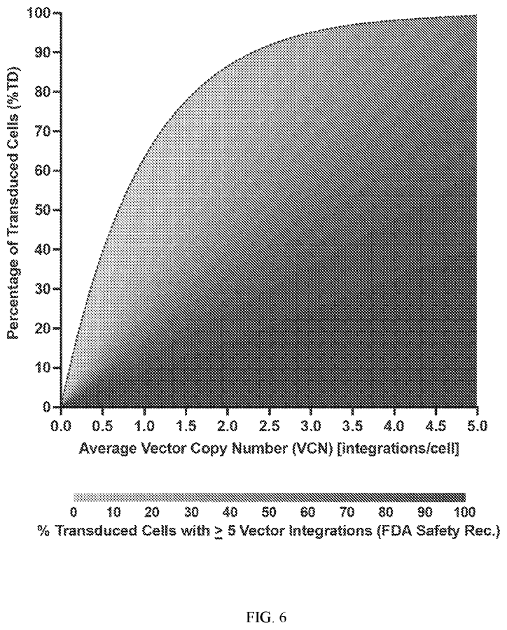

Operations in flow chart 500 may begin at block 510, a standardized relationship between transduction efficiency and average VCN with respect to integration distributions may be provided. In some examples, the standardized relationship may be received from storage. In some examples, the standardized relationship may provide a standardized space to which transduction outcomes may be compared. In some examples, the standardized relationship may be presented as a plot bounded by a curve representing the standardized relationship. The area under the curve can be divided into different zones. Each zone may represent a different safety level based on the health agency's recommendations or requirements. In some examples, the zones may be differentiated by color. FIG. 6 shows an example of a visualization of the standardized relationship/space. In some examples, the standardized relationship may be represented as a plot.

Next, at block 520, a set of the bulk transduction outcomes for each transduction of one or more transductions of one or more cell therapies or products may be received. For example, the average VCN and transduction outcomes may be received for each transduction. In some examples, a set of bulk transduction outcomes for transductions of a plurality of products may be received.

Next, at block 530, each set of the transduction outcomes may be compared to the standardized space, for example, by mapping each set of outcomes to the plot.

Next, at block 540, the visualization of the comparison of the transduction standardized relationship may be outputted. For example, a map may be stored and/or displayed.

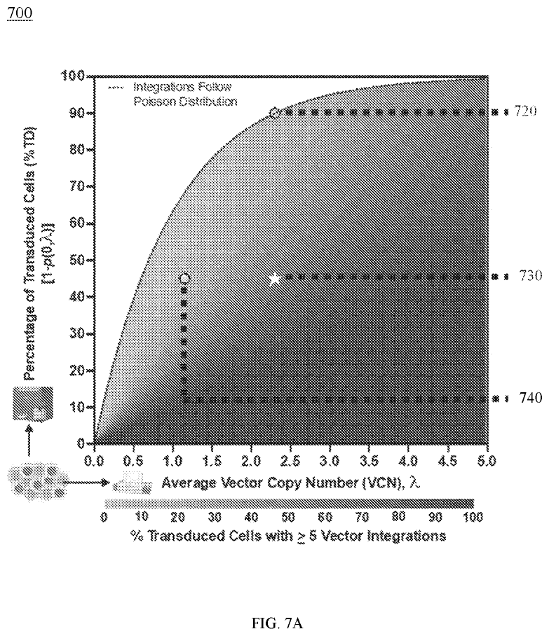

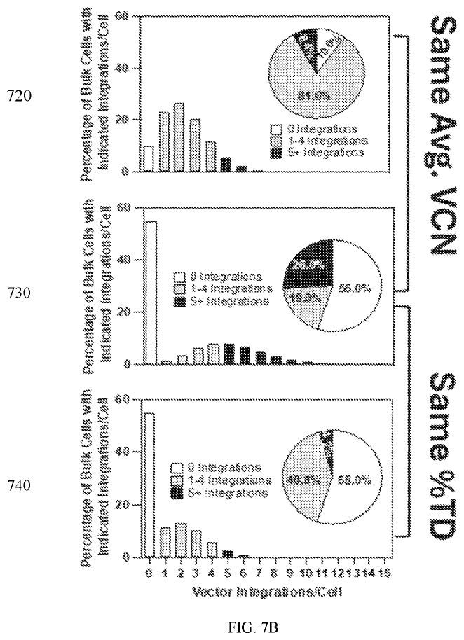

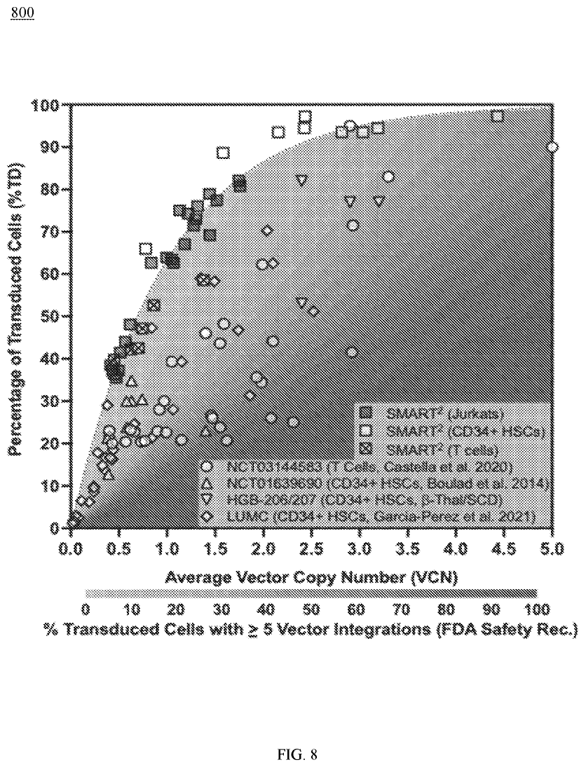

In some examples, the visualization may be used to assess safety in manufactured cell products. FIGS. 7A-10 show nonlimiting illustrative examples of the visualizations for different assessments that can be generated at block 530 and outputted at block 540. For example, FIGS. 7A and 7B show illustrative examples of the visualization of the mapped transduction outcomes according to embodiments. FIG. 8 shows another illustrative example the visualization of transduction outcomes for a plurality of cell products mapped to the standardized space.

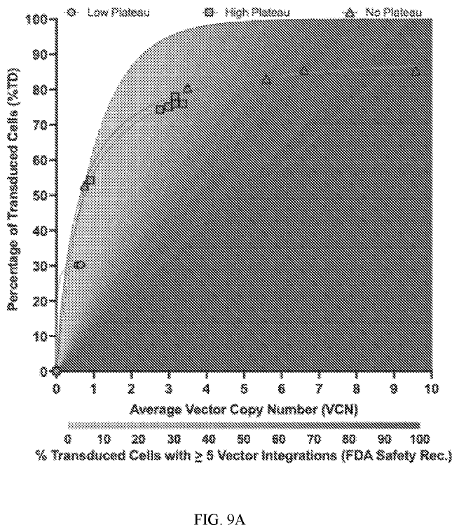

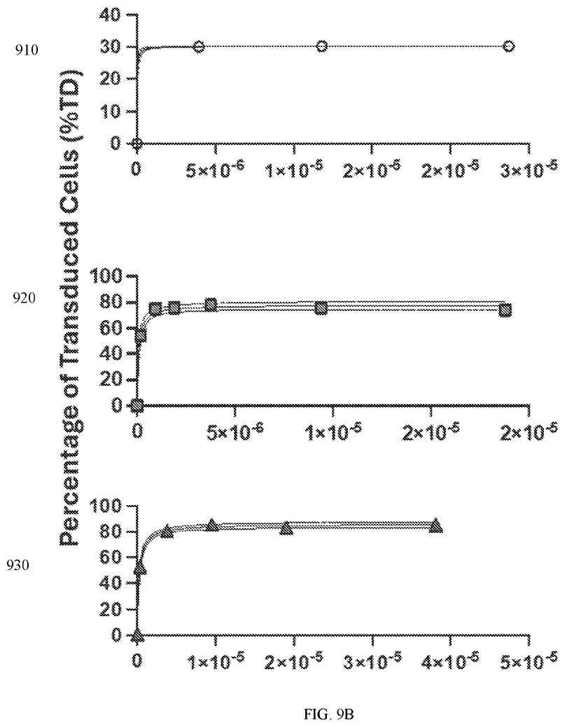

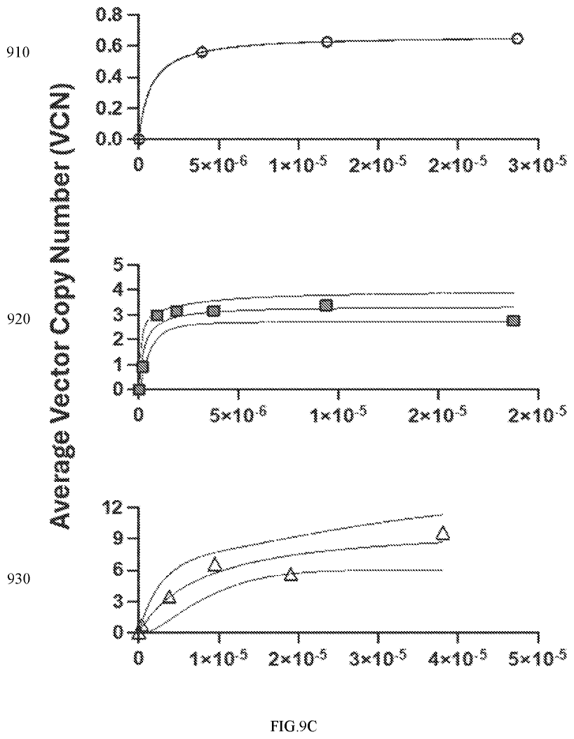

In some examples, the visualization may be used to assess integration dynamics of different vectors to characterize the viral vectors and its associated parameters for suitability for cell manufacturing. FIGS. 9A-C show illustrative examples of the visualizations according to embodiments.

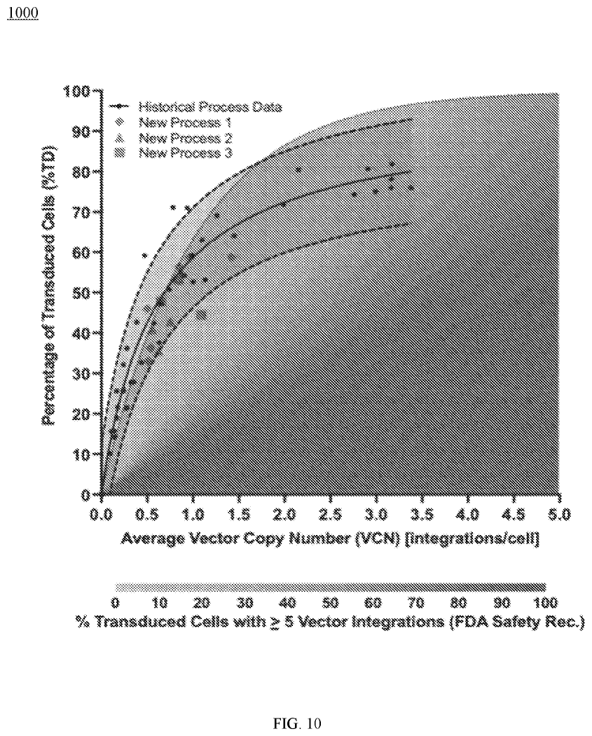

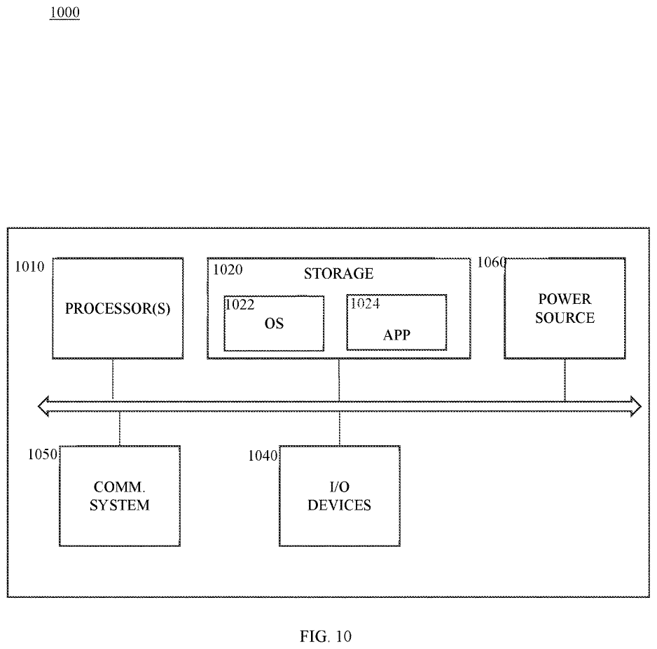

In some examples, the visualization may be used to assess comparability of different cell manufacturing processes and their impact on vector integration distribution. FIG. 10 shows an illustrative example of the visualization according to embodiments.

FIG. 6 shows an illustrative example 600 of representation of the standardized space that can be provided at block 510. In this example, the standardized space may be depicted as a plot bounded by a curve. In some examples, the curve may be generated using Poisson distribution. In this example, the curve may represent the theoretical relationship between average VCN and transduction deficiency. This may be derived using the equation % TD=100(1−e^{-average VCN}), wherein λ=average VCN. Points in the area below the curve can represent possible transduction outcomes wherein the integration distribution does not fit the expected Poisson distribution. The area below the curve may be divided into zones. The positioning and coloration of zones on the plot can be indicative of safety based on the health agency's recommendations or requirement.

FIGS. 7A and B show illustrative examples of a visualization of the standardized space and selected points within the space, respectively. FIG. 7A shows an illustrative example 700 of a plot in which the transduction outcomes are mapped. In this example, using the plot, every transduction outcome (pair of transduction efficiency and average VCN) can be evaluated for its vector integration distribution without the need to perform additional transductions. In this example, the coloring within the plot (e.g., the area under the curve) can be adjusted to indicate FDA/WHO safety recommendations for gene-modified cell therapy integrations. In this example, the coloring is based on the safety recommendations to be fewer than 5 copies/cell. The coloring can also be normalized so that only transduced cells are considered, instead of the bulk (average) VCN, which may also contain untransduced cells that lower the average VCN. In this example, the star, 730, can represent the bulk transduction outcomes from the performed transduction. In this example, the bulk transduction outcomes can be measured with flow cytometry and PCR.

FIG. 7B show visualizations of the relationship between the bulk outcomes and integrations for points 720, 730, and 740. In this example, at point 720, 8.4% of cells have 5+ integrations; and 90% of cells are transduced. According to the standardized space using these values, 9.3% of transduced cells have 5+ integrations, which is depicted by light coloring on the plot shown in FIG. 7A.

In this example, at point 730, 26% of cells have 5+ integrations; and 45% of cells are transduced. According to the standardized space using these values, 57.8% of transduced cells have 5+ integrations, which is depicted by medium coloring on the plot shown in FIG. 7A.

In this example, at point 740, 4.2% of cells have 5+ integrations; and 45% of cells are transduced. According to the standardized space using these values, 9.3% of transduced cells have 5+ integrations, which is depicted by medium coloring on the plot shown in FIG. 7A.

As shown in this example, the safety and efficacy of transduction as indicated by point 730 can be improved as demonstrated by point 720 and safety can be improved as indicated by point 740. Based on this representation, it would be beneficial to optimize the transduction parameters (e.g., viral dose, for example, according to FIGS. 1 and 2, to improve transduction outcomes, such as improving the vector integrations.

FIG. 8 shows an illustrative example 800 of a visualization generated using sets of transduction outcomes for a plurality of cell products, for example, using the method 500. In some examples, this visualization can be used to assess safety in manufactured cell products.

In this example, the outcomes of different manufactured cell products can be compared to evaluate safety (% of cells with 5+ integrations) or efficacy (% of total transduced cells). For example, the different products can be used by a company to evaluate the outcomes associated manufacturing of its own products or to compare one or more products to other manufactured products. In this example, location of point on the transduction outcomes map associated with a product's outcomes can indicate whether a process change to the manufacturing of that product may be needed to increase % transduction or lower average VCN. Based on this representation, a company could determine whether it would be beneficial to optimize the transduction parameters, for example, according to FIGS. 1 and 2, to improve transduction outcomes, such as improving the vector integrations.

FIGS. 9A-C show an illustrative example 900 of a visualization generating using sets of transductions for a plurality of different vectors, for example, using the method 500, to assess each vector for its suitability in cell manufacturing. FIG. 9A show an example 900 of an output of the visualization of mapping the bulk transduction outcomes of different viral vectors to the standardized space according to embodiments. FIG. 9B shows a visualization of the transduction efficiencies for respective viral doses associated with each vector 910, 920, 930 mapped to the output shown in FIG. 9A. FIG. 9C shows a visualization of the average vector copy number for respective viral doses associated with each vector 910, 920, 930 mapped to the output shown in FIG. 9A.

By varying a transduction parameter, such as viral dose shown in FIG. 9A, each transduction parameter can be evaluated for its suitability. In this example, the visualization can provide a standardized space in which the different integration dynamics exhibited by the different vectors can be compared. This comparison can indicate the need to change the vector manufacturing (plasmids, concentration, purification, vector design, etc.) to achieve more favorable integration dynamics. In this example, as shown in FIGS. 9A-9C, for vector 910, more vector may not further improve transduction efficiency and average VCN. Plateau may be at a lower than desirable level. For vector 920, as shown in FIGS. 9A-9C, preliminary dose dependence with transduction efficiency and average VCN result in outcomes that eventually plateau, though levels may be high enough for therapeutic usage. For vector 930, preliminary dose dependence with transduction efficiency can lead to eventual plateauing at a therapeutic level. For this vector, the average VCN exhibits dose-dependence even after % transduction plateaus, indicating that the addition of more vector will result in safety risks as transduced cells continue to take on more vector integrations.

FIG. 10 shows an illustrative example 1000 of a visualization of the mapping of transduction outcomes for a plurality of different cell manufacturing processes according to embodiments. In this example, the expected transduction integration dynamics represented from the outcomes from a previously used cell manufacturing process (represented by black circles, shaded region between dotted lines), for example, CD19-CAR LV on T cells from multiple donors with varying vector dosages resulting in different transduction outcomes, can be mapped to the standardized space. This way, the outcomes for new cell manufacturing processes (represented by different shapes) (also referred to as “candidate cell manufacturing processes”) can be juxtaposed to the historical data from the previously used cell manufacturing process within the standardized space. This allows for an assessment of comparability of outcomes of new cell manufacturing processes to expected performance (historical data).

For example, when testing cell manufacturing process changes (for example, in CAR T cell manufacturing, shortening activation time or combining activation and transduction into a single step), the transduction outcomes plot can be used to assess comparability of the integration distribution resulting from those process changes. By way of example, if the new process significantly impacted the integration dynamics, the most of the points in the visualization would populate outside of the shaded banded region of the historical process outcomes, indicating a lack of comparability for the new process.

FIG. 11 depicts a block diagram of an example computing system 1100 for implementing certain embodiments. For example, in some aspects, the computer system 1100 may include computing systems associated with a device performing one or more processes (e.g., FIGS. 1, 2, and 5) disclosed herein. The block diagram illustrates some electronic components or subsystems of the computing system. The computing system 1100 depicted in FIG. 11 is merely an example and is not intended to unduly limit the scope of inventive embodiments recited in the claims. One of the ordinary skill in the art would recognize many possible variations, alternatives, and modifications. For example, in some implementations, the computing system 1100 may have more or fewer subsystems than those shown in FIG. 11, may combine two or more subsystems, or may have a different configuration or arrangement of subsystems. In some examples, the computing system 1100 may be a part of or in communication with a cell processing system.

In the example shown in FIG. 11, the computing system 1100 may include one or more processing units 1110 and storage 1120. The processing units 1110 may be configured to execute instructions for performing various operations, and can include, for example, a microcontroller, a general-purpose processor, or a microprocessor suitable for implementation within a portable electronic device, such as a Raspberry Pi. The processing units 1110 may be communicatively coupled with a plurality of components within the computing system 1100. For example, the processing units 1110 may communicate with other components across a bus. The bus may be any subsystem adapted to transfer data within the computing system 1100. The bus may include a plurality of computer buses and additional circuitry to transfer data.

In some embodiments, the processing units 1110 may be coupled to the storage 1120. In some embodiments, the storage 1120 may offer both short-term and long-term storage and may be divided into several units. The storage 1120 may be volatile, such as static random access memory (SRAM) and/or dynamic random access memory (DRAM), and/or non-volatile, such as read-only memory (ROM), flash memory, and the like. Furthermore, the storage 1120 may include removable storage devices, such as secure digital (SD) cards. The storage 1120 may provide storage of computer readable instructions, data structures, program modules, audio recordings, image files, video recordings, and other data for the computing system 1100. In some embodiments, the storage 1120 may be distributed into different hardware modules. A set of instructions and/or code might be stored on the storage 1120. The instructions might take the form of executable code that may be executable by the computing system 1100, and/or might take the form of source and/or installable code, which, upon compilation and/or installation on the computing system 1100 (e.g., using any of a variety of generally available compilers, installation programs, compression/decompression utilities, and the like), may take the form of executable code.

In some embodiments, the storage 1120 may store a plurality of application modules 1124, which may include any number of applications, such as applications for controlling input/output (I/O) devices 1140. The application modules 1124 may include particular instructions to be executed by the processing units 1110. In some embodiments, certain applications or parts of the application modules 1124 may be executable by other hardware modules, such as a communication subsystem 1150. In certain embodiments, the storage 1120 may additionally include secure memory, which may include additional security controls to prevent copying or other unauthorized access to secure information.

In some embodiments, the storage may include an operating system 1122 loaded therein. The operating system 1122 may be operable to initiate the execution of the instructions provided by the application modules 1124 and/or manage other hardware modules as well as interfaces with the communication subsystem 1150 which may include one or more wireless or wired transceivers. The operating system 1122 may be adapted to perform other operations across the components of the computing system 1100 including threading, resource management, data storage control, and other similar functionality.

The communication subsystem 1150 may include, for example, an infrared communication device, a wireless communication device and/or chipset (such as a Bluetooth® device, an IEEE 1002.11 (Wi-Fi) device, a WiMax device, cellular communication facilities, and the like), NFC, ZigBee, and/or similar communication interfaces. The computing system 1100 may include one or more antennas (not shown in FIG. 11) for wireless communication as part of the communication subsystem 1150 or as a separate component coupled to any portion of the system.

Depending on desired functionality, the communication subsystem 1150 may include separate transceivers to communicate with base transceiver stations and other wireless devices and access points, which may include communicating with different data networks and/or network types, such as wireless wide-area networks (WWANs), WLANs, or wireless personal area networks (WPANs). A WWAN may be, for example, a WiMax (IEEE 1002.9) network. A WLAN may be, for example, an IEEE 1002.11x network. A WPAN may be, for example, a Bluetooth network, an IEEE 1002.15x, or some other types of network. The techniques described herein may also be used for any combination of WWAN, WLAN, and/or WPAN. In some embodiments, the communications subsystem 550 may include wired communication devices, such as Universal Serial Bus (USB) devices, Universal Asynchronous Receiver/Transmitter (UART) devices, Ethernet devices, and the like. The communications subsystem 1150 may permit data to be exchanged with a network, other computing systems, and/or any other devices described herein. The communication subsystem 1150 may include a means for transmitting or receiving data, such as identifiers of portable goal tracking devices, position data, a geographic map, a heat map, photos, or videos, using antennas and wireless links. The communication subsystem 1150, the processing units 1110, and the storage 1120 may together comprise at least a part of one or more of a means for performing some functions disclosed herein.

The computing system 1100 may include one or more I/O devices 1140, such as a cell processing system, a switch, a camera, a microphone or audio recorder, a communication port, or the like.

In some embodiments, the I/O devices 1140 may include a microphone or audio recorder that may be used to record an audio message. The microphone and audio recorder may include, for example, a condenser or capacitive microphone using silicon diaphragms, a piezoelectric acoustic sensor, or an electret microphone. In some embodiments, the microphone and audio recorder may be a voice-activated device. In some embodiments, the microphone and audio recorder may record an audio clip in a digital format, such as MP3, WAV, WMA, DSS, etc. The recorded audio files may be saved to the storage 1120 or may be sent to the one or more network servers through the communication subsystem 1150.

The I/O devices 1140 may also include, for example, a speaker, a media player, a display device, a communication port, or the like. For example, the I/O devices 1140 may include a display device, such as an LED or LCD display and the corresponding driver circuit. The I/O devices 1140 may include a text, audio, or video player that may display a text message, play an audio clip, or display a video clip.

The computing system 1100 may include a power device 1160 configured to power the computing system 1140.

The computing system 1100 may be implemented in many different ways. In some embodiments, the different components of the computing system 1100 described above may be integrated to a same printed circuit board. In some embodiments, the different components of the computing system 1100 described above may be placed in different physical locations and interconnected by, for example, electrical wires. The computing system 1100 may be implemented in various physical forms and may have various external appearances. The components of computing system 800 may be positioned based on the specific physical form.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

While the terms “first” and “second” are used herein to describe data transmission associated with a subscription and data receiving associated with a different subscription, such identifiers are merely for convenience and are not meant to limit various embodiments to a particular order, sequence, type of network or carrier.

Various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the claims.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing systems, (e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more example embodiments, the functions described may be implemented in hardware, software, firmware, or any combination thereof. If implemented in software, the functions may be stored as one or more instructions or code on a non-transitory computer readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, and the like.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A method for determining optimized transduction parameters, comprising:
receiving (i) specifications for one or more transduction outcomes; and (ii) bulk transduction outcomes for one or more transductions of cells with a viral vector using at least one transduction parameter;
the specifications of one or more transduction outcomes including a target number of vector integrations per cell;
the one or more bulk transduction outcomes include average vector copy number and transduction efficiency;
generating a model for each bulk transduction outcome to determine each bulk transduction outcome associated with a range of values of the at least one transduction parameter;
generating distributions of vector integrations per cell for each value of a number of values of the at least one transduction parameter using the associated modeled bulk transduction outcomes; and
determining an optimized transduction parameter for each transduction parameter from the distributions of vector integrations based on the specifications.

2. The method of claim 1, wherein:
a value of the at least one transduction parameter for each transduction being different; and
each optimized transduction parameter corresponds to a value of each modeled transduction parameter from the distributions that meets the specifications.

3. The method of according to claim 2, wherein:
the at least one transduction parameter including a dose of the viral vector.

4. The method according to claim 2, wherein:
the determining the one or more transduction parameters includes selecting a distribution of vector integrations that meets the specifications out of the distributions of vector integrations; and
each optimized transduction parameter is associated with the selected distribution.

5. The method according to claim 2, wherein the generating the model includes:
generating a model for the transduction efficiency to determine the transduction efficiency associated with a range of the at least one transduction parameter; and
generating a model for the average copy number to determine the average copy number associated with the range of the at least one transduction parameter.

6. The method according to claim 2, further comprising:
causing manufacturing of cells using the value of one or more transduction parameters.

7. The method according to claim 1, wherein the one or more bulk transduction outcomes for two or more transductions of cells with a viral vector using at least one transduction parameter are determined by transductions performed using a small-scale device.

8. The method according to claim 7, further comprising:
causing manufacturing of cells using a larger-scale device and the one or more optimized transduction parameters.

9. The method according to claim 1, wherein the bulk transduction outcomes are received for more than one cell donor and the distributions are generated for each cell donor, the method further comprising:
pooling the distributions for each cell to generate a pooled distribution;
wherein the optimized parameter is determined using the pooled distributions.

10. A method for generating a standardized visualization of transduction outcomes, comprising:
providing a standardized relationship between transduction efficiency and average VCN with respect to integration distributions;
receiving one or more sets of bulk transduction outcomes determined for one or more viral vectors and/or for one or more cell manufacturing processes;
mapping each set of bulk transduction outcomes to the standardized relationship; and
generating a visualization of the mapping.

11. The method according to claim 10, wherein the standardized relationship is depicted as a curve bound plot.

12. A system comprising:
one or more data processors; and
a non-transitory computer-readable storage medium containing instructions, which, when executed on the one or more data processors, cause the one or more data processors to perform operations including:
receiving (i) specifications for one or more transduction outcomes; and (ii) bulk transduction outcomes for one or more transductions of cells with a viral vector using at least one transduction parameter;
the specifications of one or more transduction outcomes including a target number of vector integrations per cell;
the one or more bulk transduction outcomes include average vector copy number and transduction efficiency;
generating a model for each bulk transduction outcome to determine each bulk transduction outcome associated with a range of values of the at least one transduction parameter;
generating distributions of vector integrations per cell for each value of a number of values of the at least one transduction parameter using the associated modeled bulk transduction outcomes; and
determining an optimized transduction parameter for each transduction parameter from the distributions of vector integrations based on the specifications.

13. The system of claim 12, wherein:
a value of the at least one transduction parameter for each transduction being different; and
each optimized transduction parameter corresponds to a value of each modeled transduction parameter from the distributions that meets the specifications.

14. The system of according to claim 12, wherein:
the at least one transduction parameter including a dose of the viral vector.

15. The system according to claim 12, wherein:
the determining the one or more transduction parameters include selecting a distribution of vector integrations that meets the specifications out of the distributions of vector integrations; and
each optimized transduction parameter is associated with the selected distribution.

16. The system according to claim 12, wherein the generating the model includes:
generating a model for the transduction efficiency to determine the transduction efficiency associated with a range of the at least one transduction parameter; and
generating a model for the average copy number to determine the average copy number associated with the range of the at least one transduction parameter.

17. The method according to claim 12, wherein the instructions, which, when executed on the one or more data processors, further cause the one or more data processors to perform operations including:
causing manufacturing of cells using the value of one or more transduction parameters.

18. The system according to claim 12, wherein the one or more bulk transduction outcomes for two or more transductions of cells with a viral vector using at least one transduction parameter are determined by transductions performed using a small-scale device.

19. The system according to claim 18, wherein the instructions, which, when executed on the one or more data processors, further cause the one or more data processors to perform operations including:
causing manufacturing of cells using a larger-scale device and the one or more optimized transduction parameters.

20. The system according to claim 12, wherein:
the bulk transduction outcomes are received for more than one cell donor and the distributions are generated for each cell donor; and
the instructions, which, when executed on the one or more data processors, further cause the one or more data processors to perform operations including:
pooling the distributions for each cell to generate a pooled distribution;
wherein the optimized parameter is determined using the pooled distributions.
